SEQUENCE LISTING (2 sequences)

SEQ ID NO: 1 - protein, 942 aa, Arabidopsis thaliana
MAGGGKHTPTPKAIIHQKFGAKASYTVEEVHDSSQSGCPGLAIPQKGPCLYRCHLQLPEFSVVSNVFKKKKDSEQSAAELALDKLGIRPQNDDLTVDEARDEIVGRIKYIFSDEFLSAEHPLGAHLRAALRRDGERCGSVPVSVIATVDAKINSRCKIINPSVESDPFLAISYVMKAAAKLADYIVASPHGLRRKNAYPSEIVEALATHVSDSLHSREVAAVYIPCIDEEVVELDTLYISSNRHYLDSIAERLGLKDGNQVMISRMFGKASCGSECRLYSEIPKKYLDNSSDASGTSNEDSSHIVKSRNARASYICGQDIHGDAILASVGYRWKSDDLDYDDVTVNSFYRICCGMSPNGIYKISRQAVIAAQLPFAFTTKSNWRGPLPREILGLFCHQHRLAEPILSSSTAPVKSLSDIFRSHKKLKVSGVDDANENLSRQKEDTPGLGHGFRCEVKIFTKSQDLVLECSPRKFYEKENDAIQNASLKALLWFSKFFADLDVDGEQSCDTDDDQDTKSSSPNVFAAPPILQKEHSSESKNTNVLSAEKRVQSITNGSVVSICYSLSLAVDPEYSSDGESPREDNESNEEMESEYSANCESSVELIESNEEIEFEVGTGSMNPHIESEVTQMTVGEYASFRMTPPDAAEALILAVGSDTVRIRSLLSERPCLNYNILLLGVKGPSEERMEAAFFKPPLSKQRVEYALKHIRESSASTLVDFGCGSGSLLDSLLDYPTSLQTIIGVDISPKGLARAAKMLHVKLNKEACNVKSATLYDGSILEFDSRLHDVDIGTCLEVIEHMEEDQACEFGEKVLSLFHPKLLIVSTPNYEFNTILQRSTPETQEENNSEPQLPKFRNHDHKFEWTREQFNQWASKLGKRHNYSVEFSGVGGSGEVEPGFASQIAIFRREASSVENVAESSMQPYKVIWEWKKEDVEKKKTDL

SEQ ID NO: 2 - protein, 938 aa, Oryza sativa
MPAAPTVTPKAVIHQKYGAKACYSVEEVREAVDGGCPGLALPQQTRSVYRCSLDIPGLTVVTPGTFVRKKDAEQAAAQIALDKLGIQPTANAPSTPEEAWDELIARISGFFTDENFPSSSHPLIGHMCVTFRRTGDRFGMIPMSAIAACDVKVIGLCKLIDPKAEFDPLLVLSLIYNAAKKSPGVSVSDSNFWIWSQKPYSPEAVDLALQHWSGITDPIEVDGIFVPCMMEDEPKTIRLTLSHNEHYMGDIVSKLSASDSSHAVVSRTVGKASSEIRLYFSAPNVQFVSEISHNVVSSLGDGYMESLINKRASFISGQTIYGDAILANVGYTRRDSELHTEDVTLSNYYRILLGKSPDGNYKISRDSILVAELPSVYSRSSWKGLSPRDLLCSFCRLHRLAEPYFAVNRVSASCKVLGSPVSSEEMDVLKNAENQCASDGKNDKENPDMFKCDVKIYSKKQELLLEYSTADTWSKESDAIHNSSLKVLIWFCSYFKQPNKHVLKLSHSKSTDGFTICPDNFLHEFAMFLSIYGNRGGDDSSACSTVGSLSMDTSKQKLENNAVLAHIDGPDSGVFPSHGSLTCISYTASLVVKDKTNRYTLESNNEFEFEIGTGAVKNQIESCVSQLSVNQSACFIAELPPKDLILAAANEFSHDLSKISRDNCFLEFSVKVLQVTEPLEDRMEKALFNPPLSKQRVEFAVRYINELHATTLVDFGCGSGSLLDSLLEHPTTLEKVVGVDISRKGLTRAAKSLHQKLSKKSLMQTSVPTAVLYDGSITDFDSRLYRFDIGTCLEVIEHVEEDQASLCGDVVLSSFCPTVLIVSTPNYEYNPILQRSAMPNKEEEPEENAGPCKFRNHDHKFEWTRSQFQHWATGLAEKHNYSVEFSGVGGSGDEPGFASQIAVFRRMASGQDEVCQEGELHQPYELLWEWPNASLPSH

(19) United States
(12) Patent Application Publication
(10) Pub. No.: US 2015/0259725 A1
(43) Pub. Date: Sep. 17, 2015

(54) ANALYSIS OF SMALL RNA

(71) Applicant: VILNIUS UNIVERSITY, Vilnius (LT)

(72) Inventors: Saulius Klimasauskas, Vilnius (LT); Giedrius Vilkaitis, Vilnius (LT); Aleksandra Plotnikova, Vilnius (LT)

(21) Appl. No.: 14/407,978

(22) PCT Filed: May 30, 2013
(86) PCT No.: PCT/LT2013/000009

§ 371 Date: Dec. 15, 2014

(30) Foreign Application Priority Data
Jun. 15, 2012 (GB) 1210756.1

(51) Int. Cl. C12Q 1/68

(52) U.S. Cl. CPC C12Q 1/6806

(57) ABSTRACT

A method for modifying a strand of RNA at the 3′ end, includes contacting the strand with a RNA 2′-O-methyltransferase in the presence of a co-factor, under conditions which allow for the transfer by the RNA 2′-O-methyltransferase of a part of the co-factor onto the 3′ end of the RNA strand to form a modified RNA strand, wherein the strand of RNA is included in a duplex, and wherein the part of the co-factor transferred includes a reporter group or a functional group.

### FIELD OF THE INVENTION

The present invention relates to methods of labelling RNA molecules, and to the use of these methods in the analysis of small RNA molecules in biological samples.

### BACKGROUND TO THE INVENTION

Non-coding RNAs such as miRNAs, siRNAs and piRNAs play important roles in post-transcriptional gene regulation in many species of eukaryotic organisms including humans (about 30% of all human genes along with over 60% of protein-coding genes are hypothetically regulated by microRNAs) (Friedman et al., (2009) Genome Res 19, 92-105; Lewis et al., (2005) Cell 120, 15-20; Liu and Paroo, (2010) Annu. Rev. Biochem 79, 295-319). A functional importance of small RNAs has been proven for a great variety of vital biological pathways such as development, metabolism, signal transduction, immunological response, and repression of mobile genetic elements (Bartel, (2009) Cell 136, 215-233). Overall, small RNAs maintain genome stability and integrity, and govern a wide range of human physiological and pathological processes including cancer (Croce, (2009) Nat. Rev. Genet 10, 704-714). Notably, the disease process might be controlled directly by the expression of specific miRNAs, or by reciprocal effect of miRNAs and proteins involved in pathogenesis pathway. Moreover, small RNA expression patterns are unique for different types of tissues, stem cells, disease stages or therapy responses. Thus, small RNAs have a great potential for diagnosis, prognosis and targeted development of the novel therapies for human diseases (Garofalo and Croce, (2011) Annu. Rev. Pharmacol. Toxicol 51, 25-43). Numerous experimental data demonstrate the presence of microRNAs in biological fluids (e.g. blood). Therefore inherent signature of small RNA biomarkers could be tested by non-invasive methods (Gilad et al., (2008) PLoS ONE 3, e3148; Wittmann and Jack, (2010) Biochim. Biophys. Acta 1806, 200-207).

The majority of current methods for the quantification and the analysis of known small RNAs species are based on their hybridization with oligonucleotide probes. Most often approaches based on Northern-blotting, oligonucleotide microarray technologies or reverse-transcription quantitative polymerase chain reaction (RT-qPCR) are used. However, all these strategies have inherent technical shortcomings and limitations as follows:
1. Northern-blotting measures the target RNA directly hybridized with a labeled oligonucleotide immobilized on a solid (nitrocellulose) membrane following electrophoretic separation on a polyacrylamide gel. It is relatively inexpensive and requires very basic laboratory facilities. However this method often suffers from inefficient transfer to the membrane and immobilization of short 21-24 nt RNAs. Since no target amplification is possible, relatively large amounts of input RNA sample are required for analysis.
2. Although microarray hybridization offers highly parallel analysis with multiple probes, it requires prolonged extensive experimental time, expensive equipment, advanced professional skills and large quantities of input sample. Since the methodologies are not standardized, there are significant experimental variations between different manufacturers and laboratories, often leading to considerable inconsistencies in published results (Sato et al., (2009) PLoS ONE 4, e5540).
3. RT-qPCR operates with small amounts of starting material (nanograms of total RNA). However this time-consuming and technically complicated approach is difficult to adapt for routine clinical testing. The short length of small RNAs hampers their amplification and analysis. Since the technique is not specific for the type (RNA vs. DNA) or the size of nucleic acid, there is a high probability of contamination by genomic DNA, mRNA, precursor microRNA (pri-microRNA or pre-microRNA) as well as RNA degradation products.

Current approaches used for the discovery of new species of small RNAs are based on cloning certain fractions of cellular RNA followed by their massive parallel sequencing. Such approaches share the following disadvantages:
1. The enrichment for cellular microRNAs is solely based on size-separation of single RNA strands (to 19-25 nucleotides in length) by denaturing polyacrylamide gel electrophoresis of total cellular RNA. The method is thus unable to discriminate against short degradation fragments deriving from other types of cellular RNAs (such as mRNA, rRNA etc) which inevitably leads to detection of false positive species (Frielander et al., (2008) Nat. Biotechnol 26, 407-415). This serious drawback impairs the discovery and proper analysis of new microRNAs. In addition, single-stranded RNAs are very sensitive to degradation by contaminating nucleases, and certain microRNA species may be completely lost during prolonged handling inherent for electrophoretic size-separation.
2. Endogenous priming sequences need to be attached to the 3′-termini of small RNAs by T4 RNA ligase or Poly(A)-polymerase for RT-PCR and amplification. Since both enzymes exhibit a high degree of sequence/terminal nucleotide bias, certain cellular small RNAs are often underrepresented or lost completely during cloning.

It is the aim of the present invention to solve one or of the above shortcomings of the prior art described above.

### SUMMARY OF THE INVENTION

In a first aspect the present invention provides a method for modifying a strand of RNA at the 3′ end, said method comprising contacting the strand with a RNA 2′-O-methyltransferase in the presence of a co-factor, under conditions which allow for the transfer by the RNA 2′-O-methyltransferase of a part of the co-factor onto the 3′ end of the RNA strand to form a modified RNA strand, wherein the strand of RNA is comprised in a duplex, and wherein the part of the co-factor transferred comprises a reporter group or a functional group.

The inventors have surprisingly found that RNA 2′-O-methyltransferase enzymes can direct the transfer of an extended sulfonium-bound groups from S-adenosyl-L-methionine analogs to natural miRNA and siRNA substrates from animal (including human) cells and to a variety of heteroduplexes involving RNA strands. In one embodiment the extended sulfonium-bound group comprises a reporter group that can be used directly for analysis of the RNA. In an alternative embodiment the extended sulfonium-bound group that is transferred comprises a functional group which can be utilized in a second step to bind a reporter group to the RNA strand.

Accordingly, the present invention also provides a method for analysing RNAs comprised in a biological sample, said method comprising:

(a) attaching a reporter group to the 3′, end of one or more strands of RNA in the biological sample using the method according to the first aspect of the invention;
(b) analysing the reporter group attached to the one or more strands of RNA.

Still further the present invention provides a kit for use in labeling a strand of RNA comprising in separate containers (a) a co-factor comprising a reporter group or a functional group; and (b) an RNA 2′-O-methyltransferase capable of transferring the reporter group or the functional group onto the strand of RNA when the strand is comprised in a duplex.

The methods according to the present invention can be used for the exploration and analysis of small RNAs transcriptome and the discovery of new species of microRNAs in animal and human biological samples.

In particular the methods of the present invention are advantageous because of the selectivity of the RNA 2′-O-methyltransferase enzymes towards natural small RNA strands; the size of modified RNA strand is restricted to 19-26 nucleotides with the optimal range of 21-24 nucleotides. This minimizes the possibility of undesired labelling of DNA, precursor RNAs (e.g. pri-miRNA, pre-miRNA) or degradation products of mRNAs, rRNAs, tRNAs, etc., which are also present in biological samples.

Moreover, the double-stranded nucleic acids used in the invention for the enzymatic labeling and the subsequent analysis procedures are less sensitive to nuclease contamination compared to the single-stranded RNA substrates, and therefore there is greater resistance to RNA degradation.

In view of the above, the methods of the present invention-provide an advantageous way of analyzing the entire pull of small RNAs in biological samples, and determining the presence or absence of specific small RNA molecules within a biological sample.

### DESCRIPTION OF FIGURES

The invention will be described in more detail with reference to the Figures in which:

FIG. 1 shows strategies of HEN1-directed labeling of small double-stranded RNAs in embodiments of the invention in comparison to the natural HEN1 reaction. Pathway A (left) illustrates the natural HEN1 reaction—the methyl-group transfer from S-adenosyl-L-methionine towards double-stranded RNA (R=methyl). Pathway B (centre) describes a two-step RNA labeling strategy thereby a functional group (primary amine, thiol, alkine, azide, aziridine, carboxyl, aromatic hydrocarbon, etc.) embedded in the side chain R of a synthetic cofactor is transferred to the 3′-end of each RNA strand in a RNA duplex and then the functional group is used to attach a desired reporter group in a second step. An alternative strategy C (right) depicts one-step labeling of small RNA molecules by direct HEN1-dependent transfer of a reporter group (e.g., biotin, fluorofores, etc.) embedded in the side chain R of a cofactor analog. Grey triangles represent functional groups, stars—reporter groups.

FIG. 2 shows strategies A (upper) and B (lower) for RNA 2′-O-methyltransferase-dependent analysis of native double-stranded small RNAs in biological samples in embodiments of the invention. Solid lines represent double-stranded RNAs, dotted line—single stranded nucleic acids (RNA), broken line—attached oligonucleotide adapters.

FIG. 3 shows strategies for RNA 2′-O-methyltransferase-dependent analysis of single-strand small RNAs in biological samples in an embodiment of the invention. Grey lines represent target cellular small RNA strands, dotted black line—oligonucleotide strands forming a heteroduplex with the RNA strands, broken black line—attached oligonucleotide adapters for reverse transcription and sequencing. FIG. 3A shows a strategy for analysis of small RNA of unknown sequence. FIG. 3B shows a strategy for analysis of specific RNAs in RNA pools.

FIG. 4 shows polyacrylamide gel analysis of HEN1-dependent alkylation of double-stranded RNA substrates resembling plant and animal natural microRNA. FIG. 4A. HEN1-mediated coupling of side chains on double-stranded RNA appropriated for either two-step (through primary amine from Ado-6-amine, lane 2) or one-step labeling (through Biotin reporter from Ado-biotin, lane 3) schemes. Experiments using 0.2 μM synthetic ³³P-miR173/miR173*duplex (miR173 was radiolabeled with phosphorus-33 isotope) were performed for 1 hour at 37° C. with 100 μM synthetic cofactors either in the presence of 1 μM HEN1 or in the absence of protein (lanes 1). The samples were resolved on 15% denaturing polyacrylamide gel (with 7M urea). FIG. 4B. HEN1 modified both strands of synthetic miR173/miR173* identical to natural microRNA from Arabidopsis thaliana. 0.2 μM miR173/miR173* with one reciprocally 5′-³³P-radiolabelled strand was alkylated by 1 μM HEN1 for 1 hour at 37° C. in the presence of 100 μM Ado-11-amine. FIG. 4C. Alkylation of 0.2 μM human miR-210a/miR-210a* by 1 μM HEN1 for 1 hour at 37° C. in the presence of 100 μM Ado-6-amine. Strand labeled with ³³P (to be visualized) is marked in Bold. Solid arrows point at bands corresponding to modified RNA strands; dotted arrows point at unmethylated RNA strands.

FIG. 5 shows polyacrylamide gel analysis of HEN1-dependent transfer of functional groups to unnatural RNA/DNA and RNA/LNA heteroduplexes. FIG. 5A. HEN1 covalently modifies small RNA in miRNA/DNA* duplexes. Reactions were performed using synthetic cofactor analogues with extended side chains appropriated for two-step (Ado-6-amine-radical with primary amine; lanes 2) or single-step (Ado-biotin—radical with Biotin; lane 3) labelling. 0.2 μM the hybrid heteroduplex ³³P-miR173/DNA173 with radiolabeled guide RNA strand annealed to the complementary DNA were incubated for 1 hour at 37° C. with 100 μM synthetic cofactors either in the presence of 1 μM HEN1 or in the absence of protein (lanes 1). The samples were resolved on 15% denaturing polyacrylamide gel (with 7M urea). FIG. 5B. Examination of HEN1-catalyzed alkylation of let-7a-2* duplexes with locked nucleic acids—LNA in the DNA-oligonucleotide. The alkylation reaction was performed using Ado-6-amine (lane 2) or Ado-6-ethyne (lane 3). Lane 1 represents the control sample without protein.

FIG. 6 shows polyacrylamide gel analysis of HEN1-dependent attachment of reporter groups by two-step and one-step RNA labeling mechanisms. FIG. 6A. Specific two-step labeling of RNA/DNA with fluorophore. Cy5 NHS-ester (Amersham Biosciences) is attached to the primary amino group, transferred by 2 μM HEN1 to 2 μM miR-26a*/DNA-26a* duplex from 100 μM synthetic cofactor analogue Ado-6-amine. Following the removal of excess Cy5 by RNA Clean and Concentrator-5 columns (Zymo Research), the samples were resolved on non-denaturing 12% PAGE and stained with RedSafe™ Nucleic Acid Staining Solution (iNtRON Biotechnology). Bands were visualized by FLA-5100 Image Reader (Fujifilm) using 635 nm laser for Cy5 detection (top gel) and 473 nm laser for RNA detection (bottom gel), and analyzed with MultiGauge V3.0 software. FIG. 6B. The attachment of Cy5 to duplex miR173/miR173* RNA with two-nucleotide 3′-overhangs, modified using AdoMet or Ado-11-amine. The samples were analyzed on non-denaturing polyacrylamide gel in either native state (lanes 1 and 2) or denatured by heating to 90 C in 2×RNA loading dye (Fermentas) (lanes 3 and 4). Only native double-stranded RNA was detected after ethidium bromide staining (bottom). FIG. 6C. Single-step biotinylation of miRNA using Ado-biotin. The “Input” sample was withdrawn after the modification of 0.2 μM ³³P-miR-26a*/DNA-26a* in the presence of 1 μM HEN1 and either 100 μM Ado-biotin (top gel) or 100 μM AdoMet (bottom gel), HEN1 elimination by 2 mg/ml of Proteinase K (Fermentas) in SDS-buffer (40 mM Tris-HCl at pH 7.4, 1 mM EDTA, 20 mM NaCl, 1% SDS), ethanol precipitation and the removal of excess radioactive label by Sephadex G-25 spin columns (Amersham Biosciences). The S1 is a supernatant collected after biotin-streptavidin interaction carried out in 10 mM Tris-HCl, pH 7.4, 1 mM EDTA, 2 M NaCl for 20 minutes at room temperature in the presence of Dynabeads M-270 Streptavidin (Invitrogen) in accordance to manufacturer recommendations. The S2 fraction is a supernatant collected after the first wash with 5 mM Tris-HCl, pH 7.4, 0.5 mM EDTA, 1M NaCl. The B1 fraction contains streptavidin beads resuspended in water. Solid arrow points at the RNA alkylated with Ado-biotin and/or bound to the streptavidin beads. Dotted arrow points at unmodified RNA fraction. The samples were heated with 2×RNA Loading Dye (Fermentas) and resolved on denaturing 15% PAGE with 7M urea.

FIG. 7 shows polyacrylamide gel analysis of HEN1-dependent modification of RNA/DNA substrates with different structures of RNA 3′-ends. FIG. 7A. Human miR26a is alkylated more effectively if miRNA/DNA hybrid possesses blunt-ended RNA 3′-termini. The gel on the left side shows the result of joint incubation of 0.2 μM RNA/DNA hybrid with 2nt overhangs, 1 μM HEN1 and 0.1 μM Ado-11-amine (lane 2) for 1 hour. The gel on the right depicts the alkylation of the blunt-ended hybrid (lane 4) under similar experimental conditions. Lanes 1 and 3 show the control reactions carried out in the absence of protein. Solid arrow points at the modified RNA strands; dotted arrows point at unmethylated RNA strands. The schematic view of the substrates is displayed under the gels; black lane represents RNA and grey—DNA strand. FIG. 7B. Blunt-ended RNA/DNA hybrids are completely modified using synthetic cofactors Ado-6-amine (lane 2), Ado-11-amine (lane 3) and Ado-biotin (lane 4). The comparison of lane 4 of top gel with lane 6 of FIG. 5A reveals the preference of blunt-ended substrate over the hybrid with 2-nucleotide 3′-overhangs in the alkylation reaction with Ado-biotin.

FIG. 8 shows polyacrylamide gel analysis of HEN1-dependent alkylation of target small RNA in the presence of modified oligonucleotide probes. FIG. 8A. The alkylation of RNA/DNA* duplexes with 5′-overhangs of different length. Synthetic DNA oligonucleotides used in the analysis were complementary to miR173 strand and possessed 0-7 nucleotide overhangs on their 3′-end. The synthetic imitation of the natural miR173/miR173* RNA served as negative (the first lane from left) and positive (the second lane from left) controls for the alkylation reaction. A set of 0.2 μM substrates were alkylated in the presence of 1 μM HEN1 and 0.1 μM Ado-6-amine. FIG. 8B and FIG. 8C. HEN1-mediated modification of miR173 microRNA annealed to the complementary 3′-FAM-labelled DNA strand (B) or complementary DNA oligonucleotide with internal Cy3 (C). Figure D. HEN1-mediated modification of miR-210 strand hybridized with a complementary DNA strand containing a streptavidin aptamer at its 3′-end. Solid arrows point at the bands of the modified RNA; dotted arrows point at the unmethylated RNA. Schematic view of substrates is displayed underneath the gels; black lane represents RNA, grey—the DNA strand, circle depicts the fluorophore incorporated in the DNA strand.

FIG. 9 shows polyacrylamide gel analysis of selectivity of HEN1-dependent labeling of small RNA. The functional group is attached exclusively to “target” single-stranded RNA which is complementary to the guiding. DNA. Two sets of premixed synthetic 0.2 μM of miR-26a*, let-7a-2* and miR173 in which either miR-26a* (top gel) or let-7a-2 (bottom gel) were annealed to 0.72 μM DNA complementary to miR173 (two left lanes), miR26a* (two middle lanes) or let-7a-2* (two right lanes). Then the samples were incubated for 1 hour in the presence of 1 μM HEN1 and 100 μM Ado-6-amine or in the absence of protein. Solid arrows point at the bands corresponding to modified RNA strands; dotted arrows point at unmethylated RNA strands.

FIG. 10 shows polyacrylamide gel analysis of HEN1-dependent DNA-directed labeling of specific small RNA strands in total RNA. FIG. 10A. 0.1 μM single-stranded ³³P-labelled miR173 (top gel) or let-7a-2* (bottom gel) premixed with the total RNA from E. coli in the ratio of 1:10, 1:50 and 1:100 was incubated with 0.12 μM single-stranded corresponding complementary DNA in the programmed thermostat for the re-annealing before the AdoMet was added to the mixture to a final concentration of 100 μM. Following the methylation reaction carried out for 1 hour at 37° C. in the presence of 1 μM HEN1 (or the incubation in the absence of protein—lanes on the left) the reaction mixtures were resolved on 15% denaturing polyacrylamide gel. Solid arrows point at the modified RNA bands; dotted arrows point at unmethylated RNA strands. FIG. 10B. In the similar experiment with the total RNA from U2OS cell line, the single-stranded ³³P-labelled miR173 ( 1/10 of the total RNA mixture molar concentration) was re-annealed with DNA complementary to miR173 with FAM (left panel) or standard hydroxyl group (right panel) at 3′-terminus and alkylated using Ado-11-amine.

FIG. 11 shows the strategy for, Example 3—HEN1-dependent DNA-directed modification of short RNA strands in RNA pools.

FIG. 12 shows the structure of example co-factor molecules. FIG. 12A Ado-6-ethyne. FIG. 12B Ado-6-azide. FIG. 12C Ado-6-amine and Ado-11-amine.

FIG. 13 shows the steps of Ado-biotin synthesis and the structure of Ado-biotin.

### DETAILED DESCRIPTION OF THE INVENTION

As indicated above the present invention provides in a first aspect a method for modifying a strand of RNA at the 3′ end, said method comprising contacting the strand with a RNA 2′-O-methyltransferase in the presence of a co-factor, under conditions which allow for the transfer by the RNA 2′-O-methyltransferase of a part of the co-factor onto the 3′ end of the RNA strand to form a modified RNA strand, wherein the strand of RNA is comprised in a duplex, and wherein the part of the co-factor transferred comprises a reporter group or a functional group.

### Enzyme

The present inventors have surprisingly found that RNA 2′-O methyltransferase enzymes are able to transfer a reporter group or a functional group from a co-factor onto an RNA strand in a duplex which is not the enzyme's natural substrate, and in particular is not a plant miRNA duplex.

The RNA 2′-O methyltransferase enzyme is one which is capable of binding to a duplex, e.g. the enzyme comprises a double stranded RNA binding motif. In particular, the enzyme may comprise a double stranded RNA binding domain (such as that found in the plant RNA 2′-O methyltransferase HEN1 and plant HEN1 orthologs). In particular, this domain is an N-terminal domain or in the N-terminal half of the protein. Such domains serve to stabilize the enzyme complex with the substrates (a correctly sized RNA duplex, or heteroduplex involving an RNA strand, and cofactor) and maintain their efficient interaction.

Further the enzyme is one which normally uses (or is capable of using) S-adenosyl-L-methionine (SAM or AdoMet) as a co-factor.

The biogenesis of plant miRNAs and siRNAs as well as animal piRNAs involves their modification at the 3′-termini (Kim et al., (2010) Cell 143, 703-709). In plants this reaction is carried out by a family of RNA 2′-O-methyltransferases which share a conservative catalytic domain with single-stranded RNA modifying enzymes from various eukaryotes and bacteria. HEN1 small RNA methyltransferase from Arabidopsis thaliana catalyzes the methyl group transfer from S-adenosyl-L-methionine to miRNAs and siRNAs (Yang et al., (2006) Nucleic Acids Res 34, 667-675; Vilkaitis et al., (2010) RNA 16, 1935-1942). The methylation is critical for microRNA stability in Arabidopsis since, the abundance of mature miRNAs in hen1 mutants is greatly reduced (Li et al., (2005) Curr. Biol 15, 1501-1507). Unlike certain animal homologues which modify single-stranded RNA substrates, HEN1 displays a strong preference towards duplex RNAs and efficiently methylates both strands of miRNA/miRNA* to completion (Vilkaitis et al. 2010). Plotnikova et al., previously suggested that HEN 1 may also be capable of transferring larger groups to a miR173*/miR173 plant RNA duplex (Abstracts of the 3^{rd} MC-GARD Meeting, 1-5 Apr. 2009, Cellular Oncology (2009) 111-151, P76). Although the methylation of non-plant microRNAs in vivo is fairly uncommon, the HEN1 methyltransferase is capable of appending the methyl group to animal small RNAs in vitro (Vilkaitis et al., (2010) RNA 16, 1935-1942).

The RNA 2′-O methyltransferase enzyme to be used in the method described herein may be obtained from Arabidopsis and is preferably HEN1 (obtainable from Arabidopsis thaliana), the catalytic domain of HEN1 (such as a truncated form comprising the C-terminal part of the protein (residues 666-942)) or a HEN1 homolog (or a catalytic domain thereof), such as WAVY LEAF1 (Abe et al., (2010) Plant Physiology 154, 1335-1346) obtained from rice (Oryza sativa). The sequences of the wild type HEN 1 and WAVY LEAF1 can be found in GenBank, Accession Nos. AAL05056.1 and AB583903.1, respectively. The protein sequences are as follows:

SEQ ID No: 1 HEN 1:
MAGGGKHTPT PKAIIHQKFG AKASYTVEEV HDSSQSGCPG
LAIPQKGPCLYRCHLQLPEF SVVSNVFKKK KDSEQSAAEL ALDKLGIRPQ
NDDLTVDEAR DEIVGRIKYI FSDEFLSAEH PLGAHLRAAL RRDGERCGSV
PVSVIATVDA KINSRCKIIN PSVESDPFLA ISYVMKAAAK LADYIVASPH
GLRRKNAYPS EIVEALATHV SDSLHSREVA AVYIPCIDEE WELDTLYIS
SNRHYLDSIA ERLGLKDGNQ VMISRMFGKA SCGSECRLYS
EIPKKYLDNSSDASGTSNED SSHIVKSRNA RASYICGQDI HGDAILASVG
YRWKSDDLDY DDVTVNSFYR ICCGMSPNGI YKISRQAVIA AQLPFAFTTK
SNWRGPLPRE ILGLFCHQHR LAEPILSSST APVKSLSDIF RSHKKLKVSG
VDDANENLSR QKEDTPGLGH GFRCEVKIFTKSQDLVLECSPRKFYEKEND
AIQNASLKAL LWFSKFFADL DVDGEQSCDT DDDQDTKSSS
PNVFAAPPILQKEHSSESKN TNVLSAEKRV QSITNGSWS ICYSLSLAVD
PEYSSDGESP REDNESNEEMESEYSANCES SVELIESNEE IEFEVGTGSM
NPHIESEVTQ MTVGEYASFR MTPPDAAEAL ILAVGSDTVR IRSLLSERPC
LNYNILLLGV KGPSEERMEA AFFKPPLSKQ RVEYALKHIR ESSASTLVDF
GCGSGSLLDS LLDYPTSLQT IIGVDISPKG LARAAKMLHV KLNKEACNVK
SATLYDGSIL EFDSRLHDVD IGTCLEVIEH MEEDQACEFG EKVLSLFHPK
LLIVSTPNYE FNTILQRSTP ETQEENNSEP QLPKFRNHDH KFEWTREQFN
QWASKLGKRH NYSVEFSGVG GSGEVEPGFA SQIAIFRREA SSVENVAESS
MQPYKVIWEW KKEDVEKKKT DL
SEQ ID No: 2 WAVY LEAF 1:
MPAAPTVTPKAVIHQKYGAKACYSVEEVREAVDGGCPGLALPQQTRSVYRCSLDIPGLTV
VTPGTFVRKKDAEQAAAQIALDKLGIQPTANAPSTPEEAWDELIARISGFFTDENFPSSSH
PLIGHMCVTFRRTGDRFGMIPMSAIAACDVKVIGLCKLIDPKAEFDPLLVLSLIYNAAKKSP
GVSVSDSNFWIWSQKPYSPEAVDLALQHWSGITDPIEVDGIFVPCMMEDEPKTIRLTLSH
NEHYMGDIVSKLSASDSSHAWSRTVGKASSEIRLYFSAPNVQFVSEISHNWSSLGDGY
MESLINKRASFISGQTIYGDAILANVGYTRRDSELHTEDVTLSNYYRILLGKSPDGNYKISR
DSILVAELPSVYSRSSWKGLSPRDLLCSFCRLHRLAEPYFAVNRVSASCKVLGSPVSSEE
MDVLKNAENQCASDGKNDKENPDMFKCDVKIYSKKQELLLEYSTADTWSKESDAIHNSS
LKVLIWFCSYFKQPNKHVLKLSHSKSTDGFTICPDNFLHEFAMFLSIYGNRGGDDSSACST
VGSLSMDTSKQKLENNAVLAHIDGPDSGVFPSHGSLTCISYTASLWKDKTNRYTLESNN
EFEFEIGTGAVKNQIESCVSQLSVNQSACFIAELPPKDLILAAANEFSHDLSKISRDNCFLE
FSVKVLQVTEPLEDRMEKALFNPPLSKQRVEFAVRYINELHATTLVDFGCGSGSLLDSLLE
HPTTLEKVVGVDISRKGLTRAAKSLHQKLSKKSLMQTSVPTAVLYDGSITDFDSRLYRFDI
GTCLEVIEHVEEDQASLCGDVVLSSFCPTVLIVSTPNYEYNPILQRSAMPNKEEEPEENAG
PCKFRNHDHKFEWTRSQFQHWATGLAEKHNYSVEFSGVGGSGDEPGFASQIAVFRRMA
SGQDEVCQEGELHQPYELLWEWPNASLPSH

In HEN1 the double stranded RNA binding domains are between amino acid positions 1 to 88 and 379 to 502 of SEQ ID NO: 1. The RNA 2′-O methyltransferase enzyme may comprise an RNA binding domain have at least 80%, at least 85%, at least 90%, or at least 95% sequence identity with these sections of SEQ ID NO: 1.

Preferably the RNA 2′-O methyltransferase enzyme has at, least 80% sequence identity, more preferably at least 85% sequence identity, most preferably at least 90% sequence identity with SEQ ID No: 1 or SEQ ID No: 2, or the catalytic domains of the enzymes coded by SEQ ID No: 1 or SEQ ID No: 2.

### RNA

In the method described herein the RNA strand which is modified is part of a duplex, i.e. is part of a double stranded molecule in which the RNA strand is base-paired along at least a part of its length to a second strand. In particular, the RNA strand is not miR173 from Arabidopsis.

The RNA strand is preferably a strand of animal RNA, more preferably vertebrate RNA. Accordingly, the methods of the invention can comprise an additional step of obtaining or providing a strand of RNA from a biological sample taken from an animal, preferably a vertebrate (e.g. human).

The RNA strand is 15-30 nucleotides in length, preferably 19-26 nucleotides in length, more preferably 21 to 24 nucleotides in length.

In one embodiment of the present invention the duplex contains a mismatch in a first and/or a second position of the duplex from the 3′ end, i.e. at the first and/or second nucleotide from the 3′ end of the RNA strand for which there is a corresponding position on the second strand, the first and/or second nucleotides are not properly base-paired with the corresponding position on the second strands. In this embodiment in particular the duplex may be a vertebrate miRNA/miRNA* duplex.

In one embodiment of the invention the strand of RNA is miRNA or siRNA. Preferably the miRNA or siRNA is from a biological sample taken from an animal, preferably a vertebrate. Preferably the strand of RNA is miRNA and is comprised in a miRNA/miRNA* duplex.

In an alternative aspect the strand of RNA is comprised in a duplex which is a heteroduplex, in which the nature of the second strand is different from that of the strand of RNA. In particular the second strand can also be RNA but from a different origin, e.g. the second strand can be synthetic RNA. Alternatively, the second strand can be a strand of DNA, LNA or PNA. The second strand may also contain other chemical modifications such as those that increase the stability of the heteroduplex: 2′-methyloxy, 2′-fluoro, 2′-methyloxyethyl, phosphorothioate, boranophosphate groups etc. Further suitable modifications are known in the art (see for example, “Chemical Modifications of siRNA for In Vivo Use”, Behlke, M. A., Oligonucleotides 18:305-320 (2008). Accordingly, the method of the present invention can further comprise a step of hybridizing the strand of RNA with an oligonucleotide to form the heteroduplex comprising the strand of RNA and the oligonucleotide as the second strand.

Oligonucleotides used for the hybridization can be synthesized as a random mix of defined length polynucleotides, or can be prepared with a specific sequence in order to be complementary with a small RNA of a specific sequence. As indicated in the above paragraph, the oligonucleotide can comprise a RNA, DNA, LNA strand, or other similar derivative of polynucleotides and a combination thereof. These oligonucleotides can be prepared according to methods known in the art. The oligonucleotides may be of any length but preferably are not shorter than 15 nucleotides and not longer than 60 nucleotides. More preferably the oligonucleotides are in the range from 19 nt to 54 nt (e.g. aptamers).

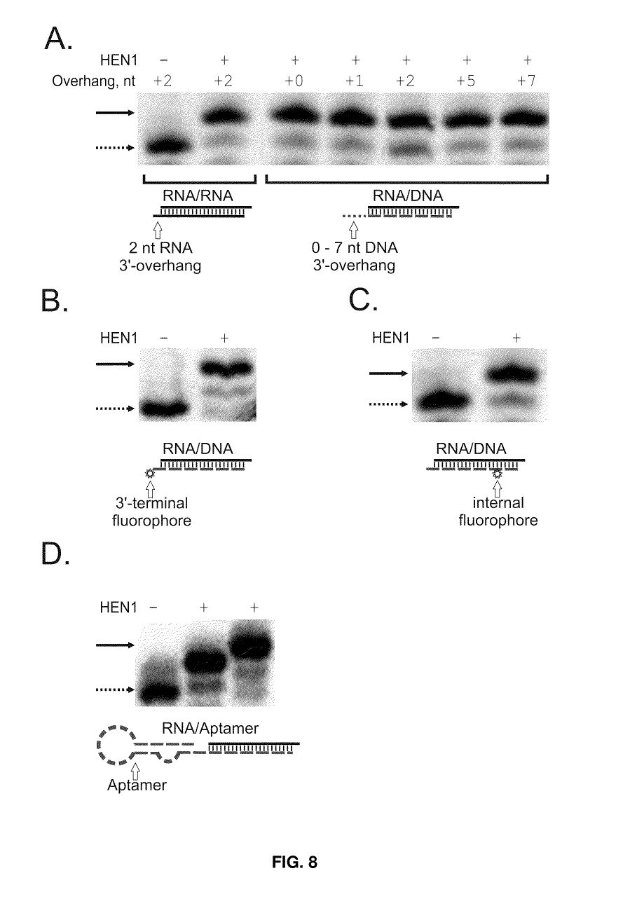

These complementary RNA, DNA or LNA oligonucleotide may contain additional functional or reporter groups both at internal and at 3′-terminal positions (for example, fluorophores, biotin, RNA and DNA aptamers, ribozymes, DNA sequences with targets for proteins or other molecules, bar codes etc.) which do not interfere with the RNA 2′-O methyltransferase-directed modification i.e. as exemplified in FIG. 8. This may lead to potentially useful applications, deriving from interactions between the reporter present on the complementary strand and the reporter group attached to the 3′ end of the strand of RNA (e.g. a FRET signal from two fluorophores).

The oligonucleotide may comprise a hairpin structure or modified nucleotides that prevent base-pairing between complementary strands etc., and which therefore limits the size of the strand of RNA to which it will base pair. As discussed below, the method of the present invention may be used to explore the small RNAs within a biological sample and therefore such a feature can be used to control the size of the strands of RNA that are present in the formed heteroduplexes.

In one embodiment of the present invention the duplex is blunt-ended at the end comprising the 3′ end of the strand of RNA. In particular, the RNA 2′-O methyltransferase is capable of modifying duplexes/heteroduplexes with both 3′ nucleotide overhangs and with blunt-ends. Blunt ended heteroduplexes can be achieved, for example, by designing synthetic DNA oligonucleotides to form blunt-ended duplexes with their target RNA strands.

### Co-Factor

The co-factor for use in the methods described herein is based on the molecule S-adenosyl-L-methionine (SAM or AdoMet), and is an S-adenosyl-L-methionine analog which comprises a functional group or a reporter group in an extended side-chain, which can be transferred onto the RNA strand by the enzyme described above.

In particular, the AdoMet analog may have the following formula:

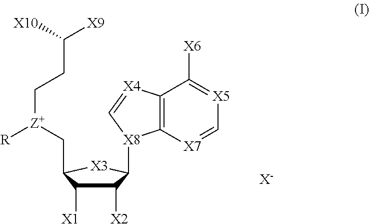

X1 and X2 represent —OH, —NH₂, —SH, —H or —F;
X3 represents —O—, —NH—, —CH₂—, —S—, or —Se—;
X4, X5, X7, X8 represent —N—, or —CH—;
X6 represents —NH₂, —OH, —OCH₃, —H, —F, —Cl, —SH or —NHCH₃;
X9 represents —CO₂H, —PO₃H, —H, —CHO, —CH₃, or —CH₂OH;
X10 represents —NH₂, —OH, —H, —CH₃, or —NHCH₃;
X⁻ is an organic or inorganic anion selected from trifluoroacetate, formate, halide and sulfonate;
Z represents S or Se;
C-bound H atoms in the adenosine moiety can be replaced by —F, —OH, —NH₂, or —CH₃;
R is the extended side-chain comprising the functional group or the reporter group.

Preferably R comprises a —CH═CH— or —C≡C— in a β-position to Z+ centre and separated therefrom by CR1 CR2-, where R1 and R2 are independently H or D.

Suitable co-factors comprising functional groups and reporter groups are also described in WO 2006/108678.

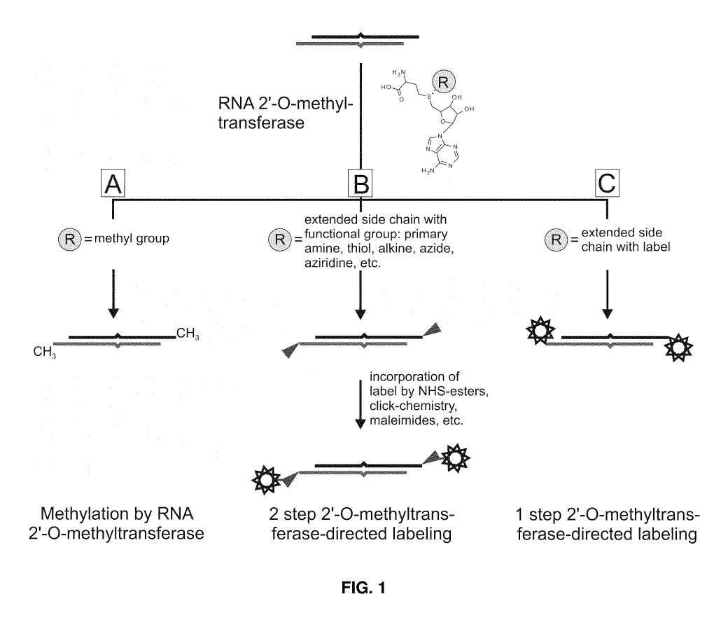

The use of a co-factor comprising a functional group or a reporter group allows for the labeling of RNA strands via two different strategies, Pathway B and Pathway C shown in FIG. 1. Pathway A (left) illustrates the natural RNA 2′-O methyltransferase reaction—the methyl-group transfer from S-adenosyl-L-methionine towards double-stranded RNA (R=methyl). Pathway B (centre) describes a two-step RNA labeling strategy whereby a functional group (primary amine, thiol, alkine, azide, aziridine, carboxyl, aromatic hydrocarbon, etc.) embedded in the side chain of a synthetic AdoMet analog is transferred to the 3′-end of each RNA strand in a RNA duplex and then the functional group is used to attach a desired reporter group in a second step. An alternative strategy C (right) depicts one-step labeling of small RNA molecules by direct RNA 2′-O methyltransferase-dependent transfer of a reporter group (e.g., biotin, fluorofores, etc.) embedded in the side chain R of a cofactor analog.

Accordingly, where the co-factor comprises a functional group, this must be capable of being used to attach a desired reporter group in a second step. In this embodiment the method of the invention may comprise a further step of reacting the functional group attached to the RNA strand with a compound comprising a reactive group (or second functional group) attached to a reporter group under conditions which allow for the transfer of the reporter group onto the RNA strand.

The functional group comprises a reactive group (group X) which may comprise an amino group, a thiol group, a hydrazine group, a hydroxylamine group, a 1,2-aminothiol group, an azide group, a diene group, an alkyne group, an arylhalide group, a terminal silylalkyne group, an N-hydroxysuccinimidyl ester group, a thioester group, an isothiocyanate group, an imidoester group, a maleimide group, a haloacetamide group, an aziridine group, an arylboronic acid group, an aldehyde group, a ketone group, a phosphane ester group, a dienophile group, or a terminal haloalkyne group.

Group X can then be reacted with a compound comprising a second reactive group (group Y) which is attached to the reporter group. Suitable groups for X and Y, and the subsequent linkage which the reaction forms between the RNA strand and the reporter group are shown below in Table 1. Suitable reporter groups are a fluorophore, a quantum dot, an oligonucleotide primer, a DNA aptamer, a RNA aptamer, a ribozyme, DNA with specific protein targets or sequences for analysis (bar codes), or an affinity tag (which are discussed further below).

TABLE 1
Mutually reactive functional groups X and Y suitable for conjugation

| Reactive group X or Y | Reactive group Y or X | Linkage formed |
|---|---|---|
| Primary amine | N-hydroxysuccinimidyl | amide |
| | ester | |
| Primary amine | thioester | amide |
| Primary amine | isothiocyanate | thioureas |
| Primary amine | imidoester | imidate |
| Primary amine | aldehyde, ketone | imine/secondary |
| | | amine after reduction |
| Thiol | maleimide | thioether |
| Thiol | haloacetamide | thioether |
| Thiol | aziridine | thioether |
| Thiol | thiol | disulfide |
| Hydrazine | aldehyde, ketone | hydrazone |
| Hydroxylamine | aldehyde, ketone | oxime |
| 1,2-Aminothiol | aldehyde, ketone | thiazolidine |
| 1,2-Aminothiol | thioester | amide |
| Azide | alkyne | 1,2,3-triazole |
| Azide | phosphane ester | amide |
| Diene | dienophile | cyclohexene |
| Terminal alkyne | arylhalide | arylalkyne |
| Arylhalide | arylboronic acid | biaryl |
| Terminal silylalkyne | terminal haloalkyne | diyne |

The functional group of the co-factor may optionally be in protected form, such as a protected amino group, a protected thiol group, a protected hydrazine group, a protected hydroxyamino group, a protected aldehyde group, a protected ketone group and a protected 1,2-aminothiol group. As such, the reactive group X may be first transferred from the co-factor to the RNA strand in a protected form as a derivative that is converted to an active functional form in a separate step. For example, thiols may be transferred with acetyl protecting group (protected F1=—S—COCH₃) which can be readily removed to yield thiol (F1=—SH) by treatment of modified DNA with 20% ammonia, or transferred 1,2-diol can be converted to aldehyde by oxidation with sodium periodate.

Alternatively, in a one-step labeling procedure the extended side-chain R of the AdoMet analog comprises a reporter group. Suitable reporter groups include a fluorophore, a quantum dot, an affinity tag, an oligonucleotide primer, a DNA aptamer, an RNA aptamer, ribozymes, or DNA with specific protein targets or sequences for analysis (bar codes). The affinity tag may be biotin, maltose, c-myc-tag, HA-tag, digoxygenin, flag-tag, dinitrophenol, His tag, strep-tag, glutathione, or nickel-nitrilotriacetic acid (NTA).

### Analysis Methods

As indicated above, the method of the present invention has particular utility in analysis of the small RNA in biological samples, including the determination of the types of small RNA present in a particular sample, and the exploration and discovery of new species of small RNA within the small RNAs transcriptome in biological samples. Accordingly, the method described above can further comprise steps of using the reporter groups or functional groups to enrich, to clone and/or to sequence the RNA strand, to detect or quantitate the small RNAs.

In particular, the method of the present invention can be used in the analysis of both native double-stranded small RNAs and single-stranded small RNAs in a biological sample. Accordingly, all of the methods described herein may comprise a step of obtaining and/or preparing a biological sample. In particular, the biological sample can be individual cells, cultured cells, tissues (highly differentiated, fetal), biopsy, bodily fluids (e.g. blood, urine, tears, saliva). Methods of preparing such samples so that they are suitable for analysis of the small RNAs they contain are known in the art.

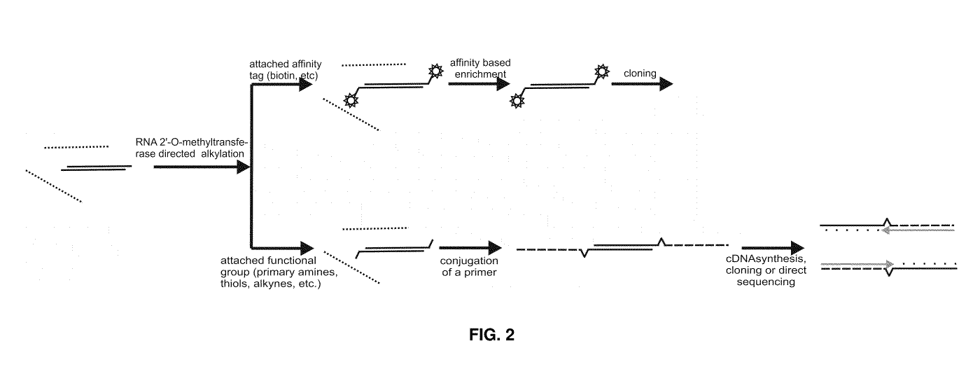

In a particular embodiment the method of the present invention is used to examine the double-stranded small RNA pool (especially miRNA) from a biological sample by the strategies shown in FIG. 2: RNA 2′-O methyltransferase-dependent alkylation to attach affinity reporter (e.g. biotin) for selective enrichment and cloning (A) or RNA 2′-O methyltransferase-dependent alkylation to attach an oligonucleotide adapter for primed reverse transcription and sequencing (B).

This method of strategy A can comprise the steps of:
(a) labeling the 3′ end of both RNA strands in a RNA duplex in the biological sample using the methods described above to attach an affinity group to both strands;
(b) affinity binding and enrichment of the labeled RNA strands; and
(c) cloning and sequencing of the enriched RNA strands.

The method of strategy B can comprise the steps of
(a) attaching a functional group to the 3′ end of both RNA strands in a RNA duplex in the biological sample using the methods described above;
(b) reacting the functional group with a reactive group attached to an oligonucleotide under conditions that allow for the transfer of the oligonucleotide onto the 3′ end of both RNA strands;
(c) sequencing the RNA strands using the oligonucleotide.

To the extent that the RNA 2′-O-methyltransferase modifies both strands of the duplex, the (massive parallel) sequencing would detect both the guide (functional) and the passenger (*) microRNA strands. Due to close genomic location of these sequences, the analysis provides the beneficial supplementary data for the precise bioinformatics predictions and mapping of the microRNA genes.

Even though some cellular microRNAs are thought to exist in single-stranded form (which are not modified by the RNA 2′-O-methyltransferase), the method of the invention can be successfully applied for the discovery of new non-coding RNAs after induction of a small RNA maturation arrest, which leads to the accumulation of double-stranded miRNA/miRNA*. This effect is achieved by viral infection of cell cultures or by addition of inhibitors of the Argonaute protein. Accordingly, the method of the present invention can further comprise the step of inducing small RNA maturation arrest in the biological sample, e.g. in animal cell cultures or even in whole organisms in lower animals (nematodes). Preferably the step of inducing small RNA maturation arrest comprises adding an Argonaute protein inhibitor to the biological sample, or infecting the cell culture with a virus. This approach allows the detection of cellular microRNAs, whose expression level affected in response to a treatment or environmental conditions. Simultaneous changes in the environmental context and the sequestration of miRNA/miRNA* processing towards the mature single-stranded form may lead to the stimulus-induced accumulation of the particular microRNA/microRNA* species. It is important to note that already existed cellular single-stranded microRNAs expressed prior the treatment remain unlabeled. (Therefore, comparison of treated and untreated samples may assess the nature and time span of microRNA alterations.)

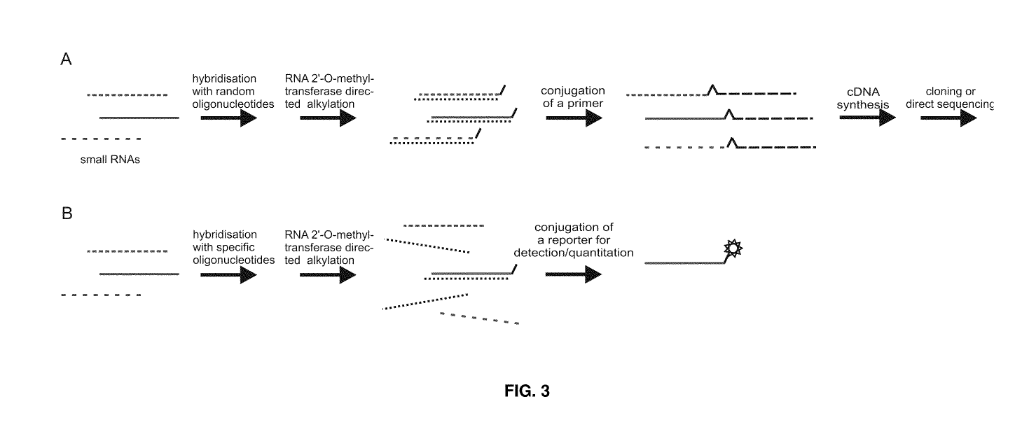

Further methods of the invention involve RNA 2′-O-methyltransferase-dependent modification of a single-strand of small RNAs within a heteroduplex in order to examine the native single-stranded RNA pool taken from a biological sample. In particular, the limited amount of double-stranded microRNAs in biological samples can impede the detection of cellular microRNAs without the arrest of small RNAs maturation. This problem is solved by the hybridization of small RNA pool with a random oligonucleotide probes, as shown in FIG. 3.

This method can comprise the steps of:
(a) contacting a biological sample comprising one or more RNA strands with random oligonucleotides under conditions which allow for the hybridization of one or more RNA strands with the oligonucleotides to form one or more heteroduplexes;
(b) labeling the 3′ end of a RNA strand in a heteroduplex in the biological sample using the methods described above;
(c) affinity binding and enrichment of the labeled RNA strands; and/or cloning and sequencing of the RNA strands.

The oligonucleotides used for the hybridization can be synthesized as a random mix of defined length polynucleotides, for example, a 22-mer of any bases.

Further, in order to restrict the length of the explored RNAs, the randomly synthesized oligonucleotides can be linked to the hairpin structure or modified nucleotides that prevent base-pairing between complementary strands etc.

The method involving the formation of heteroduplexes can also be used for the detection and quantification of known small RNA in biological samples. Such a method utilizes oligonucleotides with sequences which are complementary to the RNA which it is desired to detect (i.e. locus-specific oligonucleotide probes). Such a method to determine the presence of an RNA molecules in a sample involves the steps of:
(a) contacting the sample with an oligonucleotide probe under conditions which allow for the hybridization of the oligonucleotide to the RNA molecule to form a heteroduplex;
(b) labeling of the RNA strand of the heteroduplex with a suitable reporter group using the methods described herein;
(c) detection the presence of the reporter group so as to determine the presence or absence of the RNA molecule in the sample, and optionally quantifying the reporter group so as to determine the amount of the RNA molecule in the sample.

The above method can be multiplexed using a number of specific oligonucleotide probes in the same reaction.

As indicated above, the oligonucleotide probe (OP) can comprise a RNA, DNA, LNA strand, or other similar derivative of polynucleotides and a combination thereof.

The oligonucleotide probe may contain additional chemical moieties, as described above, such as fluorophores, affinity binders, aptamers, ribosymes, targets for functional proteins etc. Additional chemical moieties may be attached at the 3′terminus or internally.

The detection of the attached reporter on the RNA strand can be achieved by the emission of fluorescence, or assays specific for the reporter group being used (e.g. avidin or streptavidin conjugated to peroxidase or alkaline phosphotase, antigens, aptamers, color-codes tiny beads—microsphere particles, beads etc).

As moderate 3′-extensions of the OP strand do not interfere with RNA 2′-O-methyltransferase-mediated reaction, oligonucleotide probes with functional nucleic acids or groups (e.g., aptamers, ribosymes, and targets for (reporter) proteins suitable for sensing) can be used.

Internal covalent modifications in the probe sequences also do not impair the RNA 2′-O-methyltransferase-dependent reaction. Accordingly, probes can be designed with suitable donor-acceptor distances between a fluorophore in a guide oligonucleotide and the reporter group (transferred by the method of the invention) on the 3′-terminal group on the target RNA that permit efficient fluorescence resonance energy transfer (FRET). This can be used for FRET based detection and quantitation of selected small RNAs in biological samples.

The present invention allows the development of tools for clinical diagnostics based on simultaneous quantitation of several types small RNAs. Although different types and stages of diseases including cancer or virus infections are characterized by unique signature of biomarkers (changes in miRNA levels), the expression levels of the majority of small RNAs remain constant. Therefore multiplex format may be more be beneficial in terms of speed and cost over global analysis of miRNA levels.

RNA 2′-O-methyltransferase-dependent RNA labeling allows developing a set of tools for research and clinical applications: analysis and detection in fluid systems, solid substrates, in situ, by confocal fluorescence microscopy, etc.

### Kit

In a further aspect the present invention provides a kit for use in labeling a strand of RNA comprising (a) a co-factor comprising a reporter group or a functional group; and (b) an RNA 2′-O-methyltransferase capable of transferring the reporter group or the functional group onto the strand of RNA when the strand is comprised in a duplex.

The kit may further comprise (c) a set of oligonucleotides having a random sequence or a single oligonucleotide or a set of oligonucleotides of specific sequence, which are designed to hybridise to a specific small RNA. The oligonucleotides are described in detail above, but in particular may optionally contain additional chemical modifier groups or reporter groups.

Each element of the kit is in a separate container.

The kit may optionally further comprise instructions for using the components of the kit in order to label a strand of RNA. The instructions are provided on an appropriate medium, for example paper or an electronic data carrier.

The description herein regarding the methods of the present invention also applies to the elements of the kit of the invention.

The present invention will now be described in further detail, by way of example only, with reference to the following Examples and related Figures.

### EXAMPLES

### Example 1

### HEN1-Dependent Modification of Short miRNA Duplexes Containing Two-Nucleotide 3′-Overhangs

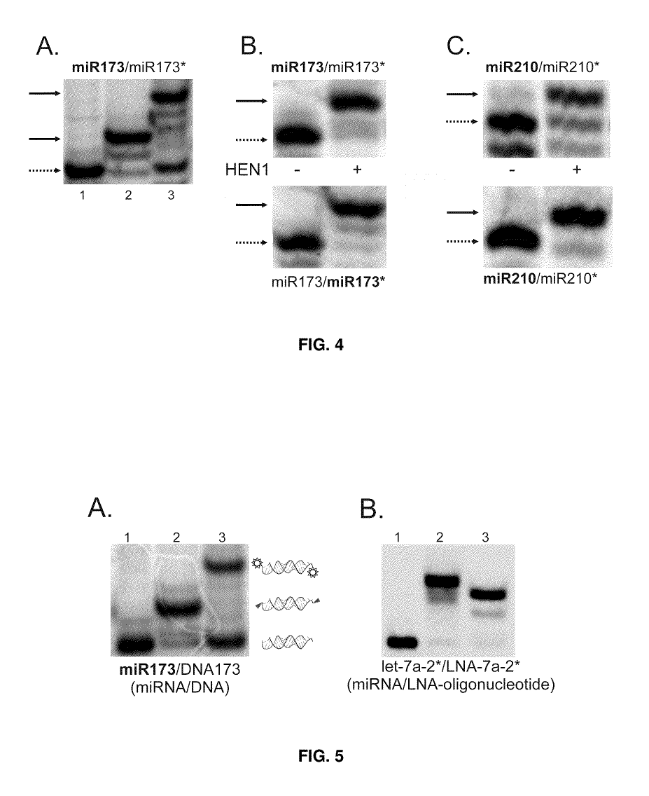

The results of Example 1 are shown in FIG. 4, which demonstrates HEN1-dependent alkylation of double-stranded RNA substrates (miR173/miR173* and miR210/miR210*) resembling plant and animal natural microRNA. In these gel photographs the strand labeled with ³³P (to be visualized) is marked in Bold. Solid arrows point at bands corresponding to modified RNA strands; dotted arrows point at unmethylated RNA strands.

In FIG. 4A it was demonstrated that HEN1-mediated coupling of side chains on double-stranded RNA was appropriate for either two-step (through primary amine from Ado-6-amine, lane 2) or one-step labeling (through Biotin reporter from Ado-biotin, lane 3) schemes. Experiments using 0.2 μM synthetic ³³P-miR173/miR173*duplex (miR173 was radiolabeled with phosphorus-33 isotope) were performed for 1 hour at 37° C. with 100 μM synthetic cofactors either in the presence of 1 μM HEN1 or in the absence of protein (lanes 1). The samples were resolved on 15% denaturing polyacrylamide gel (with 7M urea).

In FIG. 4B it was demonstrated that HEN1 modified both strands of synthetic miR173/miR173* identical to natural microRNA from Arabidopsis thaliana. 0.2 μM miR173/miR173* with one reciprocally 5′-³³P-radiolabelled strand was alkylated by 1 μM HEN1 for 1 hour at 37° C. in the presence of 100 μM Ado-11-amine.

In FIG. 4C alkylation of 0.2 μM human miR-210a/miR-210a* by 1 μM HEN1 was performed for 1 hour at 37° C. in the presence of 100 μM Ado-6-amine.

Accordingly, RNA duplexes resembling plant and animal miRNA can be covalently modified at their 3′-termini with extended groups in a HEN1-dependent manner.

### Example 2

### HEN1-Dependent Modification of RNA Strands in Short Heteroduplex Substrates

The results of Example 2 are shown in FIGS. 5, 6, 7 and 8, which demonstrate the ability of the enzyme HEN1 to transfer a modified group (either a functional group or a reporter group) to unnatural RNA/DNA and RNA/LNA heteroduplexes.

The gel photos in FIG. 5 show the result of the modification of RNA/DNA (FIG. 5A) and RNA/LNA (FIG. 5B) with two-nucleotide 3′ overhangs, with HEN1.

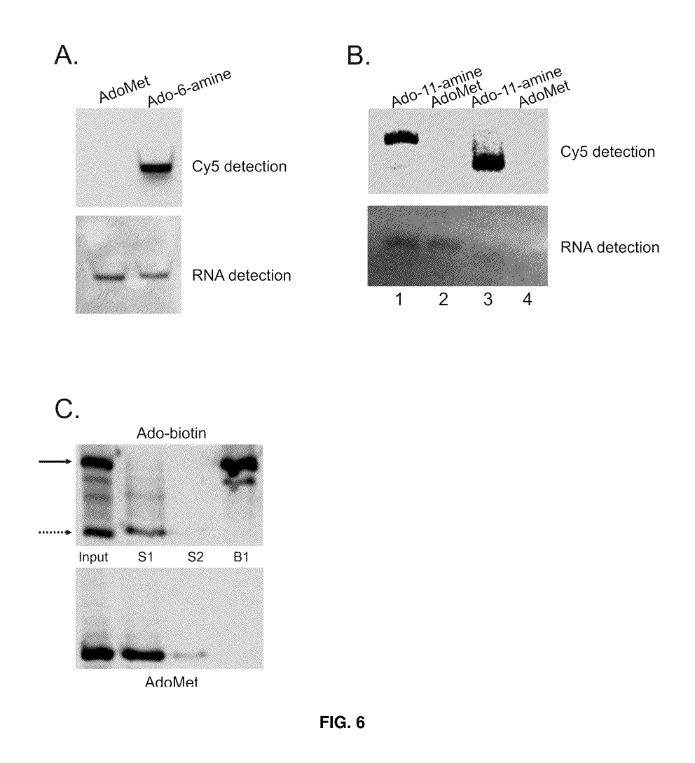

Further results from experiments with RNA/DNA heteroduplexes are shown in FIGS. 6 to 8. In particular, FIG. 6A shows the two-step labeling of miR-26a*/DNA-26a*. A similar covalent two-step labeling of an miRNA/miRNA* duplex with a fluorophore is shown in FIG. 6B, FIG. 6C demonstrates the one-step labeling of RNA strands in an RNA/DNA heteroduplex with biotin.

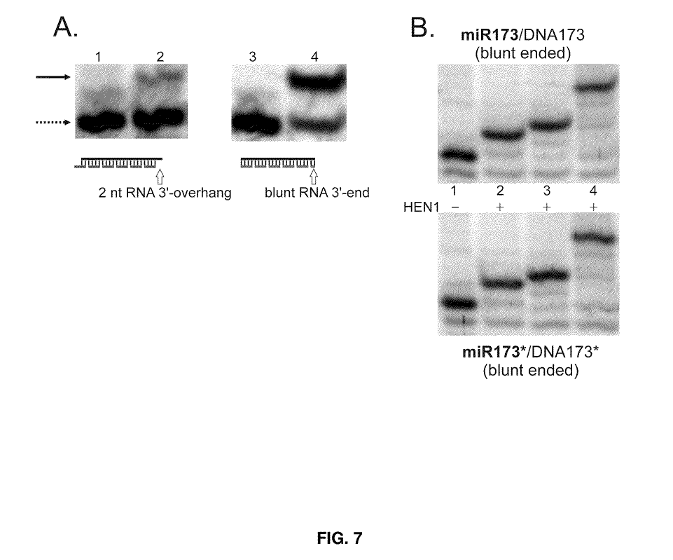

FIG. 7 provides results of experiments demonstrating that modification and one-step labeling of RNA strands can be directed to RNA/DNA heteroduplexes that contain no 3′-overhangs. In FIG. 7A it is demonstrated that human miR26a is modified more effectively if the miRNA/DNA hybrid contains blunt-ended 3′ termini. FIG. 7B demonstrates that blunt-ended RNA/DNA hybrids are completely modified using a range of synthetic cofactors.

FIG. 8 shows HEN1-dependent modification of RNA strands in RNA/DNA heteroduplexes whose DNA strand carries various 3′-terminal or internal extensions. In particular, FIG. 8A shows the modification of RNA/DNA* duplexes with overhangs of different lengths. FIG. 8B and FIG. 8C show the modification of RNA/DNA duplexes with a 3′ terminal fluorophone (FIG. 8B) and an internal fluorophore (FIG. 8C) in the DNA strand. FIG. 8D shows that modification still occurs when the DNA strand in the RNA/DNA duplex contains a streptavidin aptamer at its 3′end.

### Example 3

### HEN1-Dependent DNA-Directed Modification and Labeling of Short RNA Strands in RNA Pools

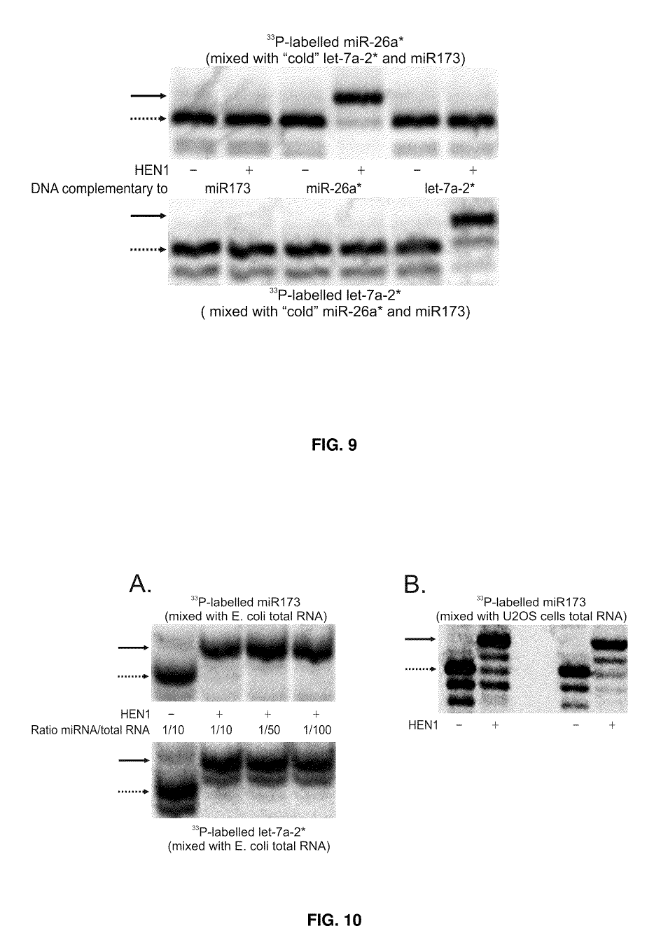

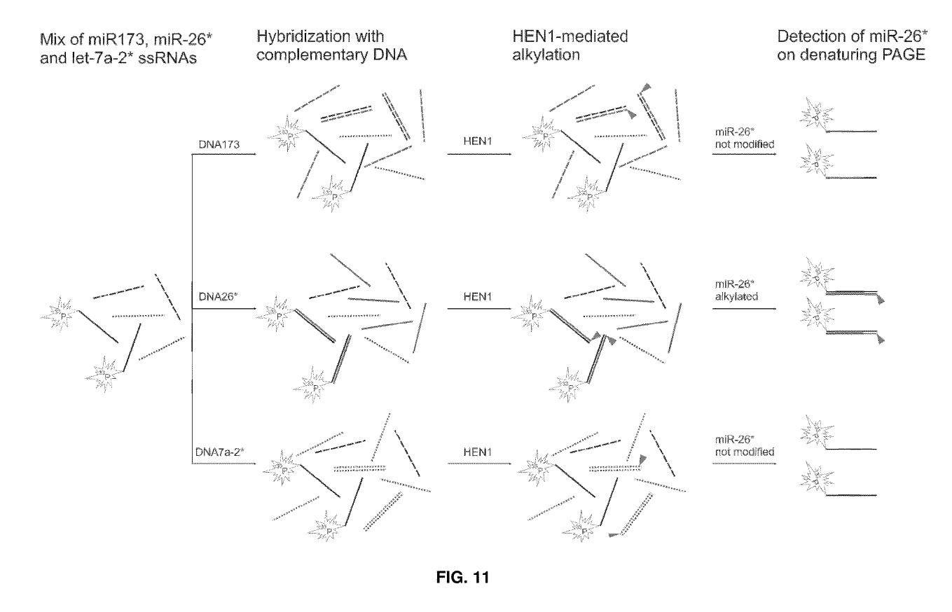

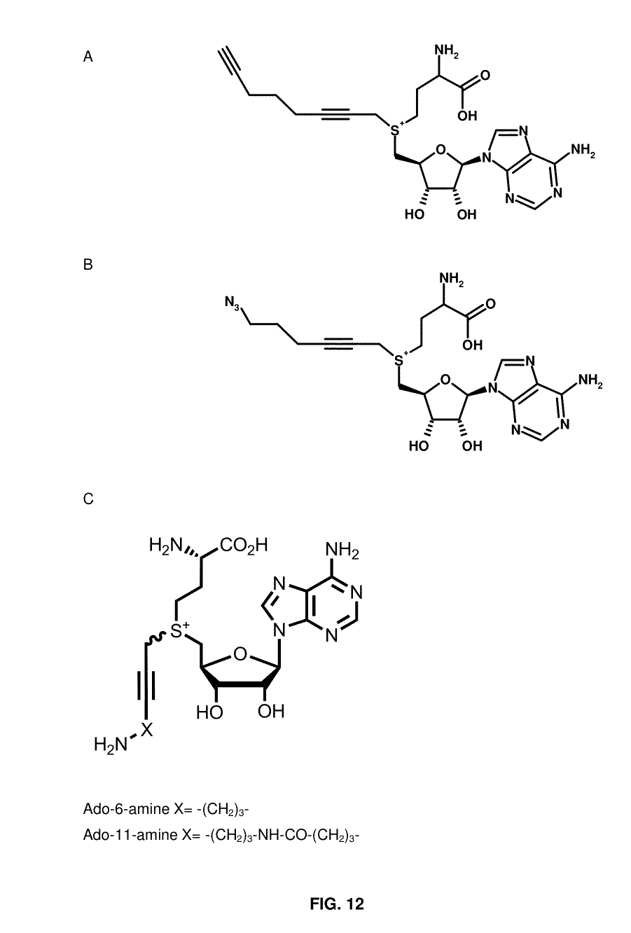

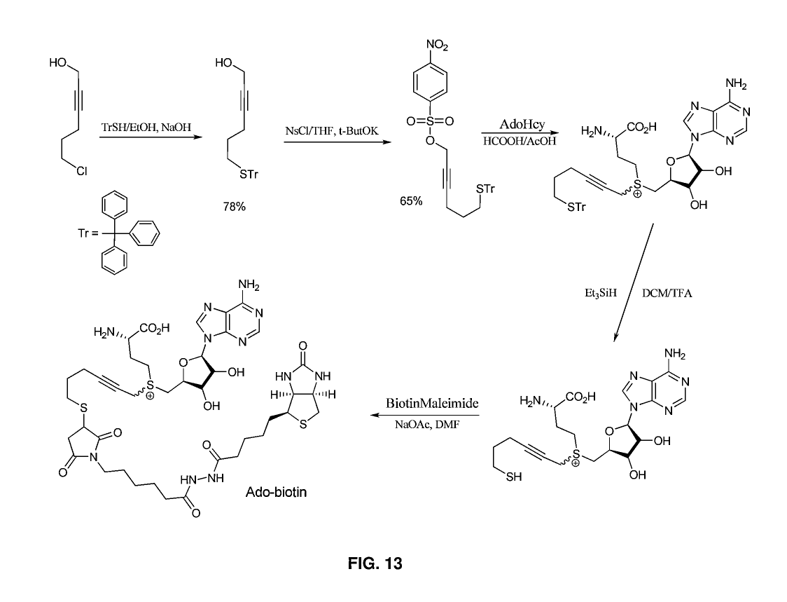

Example 3 demonstrates HEN1-dependent DNA-directed modification of short RNA strands in RNA pools, the experimental strategy for which is shown in FIG. 11. The results are shown in FIGS. 9 and 10. FIG. 9 shows the modification of miR173, miR-26a* and let-7a-2** by HEN1 after hybridization to complementary DNA. This demonstrates that 2′-O-methyltransferase-dependent modification can be directed by a DNA probe to a specified RNA strand in the presence of other RNAs resembling of plant and animal RNA.

FIG. 10 shows that 2′-O-methyltransferase-dependent labeling can be selectively directed by a DNA probe to a specific RNA strand in the presence of total cellular RNA of bacterial or animal origin. In FIG. 10A single stranded miR173 or let-7a-2* is premixed with total RNA from E. coli in the presence of complementary DNA and the ability of HEN1 to modify the miR173 or let-7a-2* strands using AdoMet as a cofactor is demonstrated. In FIG. 10B single stranded miR173 is premixed with total RNA from U2OS cell line in the presence of complementary DNA and the ability of HEN1 to modify the miR173 using a synthetic co-factor is demonstrated.

1-36. (canceled)

37. A method for modifying a strand of RNA of 19 to 26 nucleotides in length at the 3′ end, said method comprising contacting the strand with a RNA 2′-O-methyltransferase in the presence of a co-factor, under conditions which allow for the transfer by the RNA 2′-O-methyltransferase of a part of the co-factor onto the 3′ end of the RNA strand to form a modified RNA strand, wherein the strand of RNA is comprised in a duplex, and wherein the part of the co-factor transferred comprises a reporter group or a functional group.

38. A method according to claim 37 wherein the duplex contains a mismatch in a first and/or a second position of the duplex from the 3′ end of the RNA strand.

39. A method according to claim 37 wherein the strand of RNA is miRNA or siRNA.

40. A method according to claim 37 wherein the strand of RNA is comprised in a duplex which is a heteroduplex.

41. A method according to claim 40 wherein the heteroduplex comprises the strand of RNA and a strand of DNA, LNA or PNA, synthetic RNA.

42. A method according to claim 41 wherein the strand of DNA, LNA, PNA or synthetic RNA comprises a chemical modification to increase the stability of the heteroduplex and/or a second functional or reporter group.

43. A method according to claim 37 wherein the duplex is blunt-ended at the end comprising the 3′ end of the strand of RNA.

44. A method according to claim 37 wherein the method further comprises a step of forming the duplex by contacting the strand of RNA with a complementary strand.

45. A method according to claim 37 wherein the RNA 2′-O-methyltransferase comprises an amino acid sequence having at least 80% sequence identity with SEQ ID No: 1.

46. A method according to claim 37 wherein the co-factor is an S-adenosyl-L-methionine analog.

47. A method according to claim 37 wherein the co-factor comprises a functional group which is an amino group, a thiol group, a hydrazine group, a hydroxylamine group, a 1,2-aminothiol group, an azide group, a diene group, an alkyne group, an arylhalide group, a terminal silylalkyne group, an N-hydroxysuccinimidyl ester group, a thioester group, an isothiocyanate group, an imidoester group, a maleimide group, a haloacetamide group, an aziridine group, an arylboronic acid group, an aldehyde group, a ketone group, a phosphane ester group, a dienophile group, and a terminal haloalkyne group.

48. A method according to claim 37 wherein the co-factor comprises a functional group and the method further comprises a step of reacting the functional group with a compound comprising a reporter group under conditions which allow for the transfer of the reporter group to the RNA strand.

49. A method according to claim 37 wherein the reporter group is a fluorophore, a quantum dot, an affinity tag, an oligonucleotide, a DNA aptamer, an RNA aptamer.

50. A method according to claim 49 wherein the affinity tag is biotin, c-myc-tag, HA-tag, digoxygenin, flag-tag, dinitrophenol, His tag, strep-tag, glutathione, nickel-nitrilotriacetic acid (NTA), or maltose.

51. A method for analysing RNAs comprised in a biological sample, said method comprising:
(a) attaching a reporter group to the 3′ end of one or more strands of RNA in the biological sample using the method of claim 37;
(b) analysing the reporter group attached to the one or more strands of RNA.

52. A method according to claim 51 wherein the reporter group is an affinity tag and step (b) comprises affinity binding and enrichment of the RNA strands attached to the affinity tag.

53. A method according to claim 52 further comprising the step of cloning and sequencing the enriched RNA strands.

54. A method according to claim 51 wherein the reporter group is an oligonucleotide primer and step (b) comprises sequencing of the one or more strands of RNA to which the oligonucleotide primer is attached.

55. A method according to claim 51 wherein step (b) comprises detecting and quantifying the reporter group attached to the one or more strands of RNA.

56. A method according to claim 51 comprising a step of providing the biological sample, wherein the biological sample is prepared from a cell culture.

57. A method according to claim 56 wherein the preparation includes a step of inducing small RNA maturation arrest, which comprises adding an Argonaute protein inhibitor to the biological sample, or infecting the cell culture with a virus.

58. A kit for use in labeling a strand of RNA, comprising in separate containers (a) a co-factor comprising a reporter group or a functional group; and (b) an RNA 2′-O-methyltransferase capable of transferring the reporter group or the functional group onto the strand of RNA when the strand is comprised in a duplex, wherein the co-factor comprises a functional group which is an amino group, a thiol group, a hydrazine group, a hydroxylamine group, a 1,2-aminothiol group, an azide group, a diene group, an alkyne group, an arylhalide group, a terminal silylalkyne group, an N-hydroxysuccinimidyl ester group, a thioester group, an isothiocyanate group, an imidoester group, a maleimide group, a haloacetamide group, an aziridine group, an arylboronic acid group, an aldehyde group, a ketone group, a phosphane ester group, a dienophile group, and a terminal haloalkyne group.

59. A kit according to claim 58 wherein the co-factor comprises a reporter group and the reporter group is a fluorophore, a quantum dot, an oligonucleotide primer, a DNA aptamer, an RNA aptamer, or an affinity tag.

60. A kit according to claim 58 wherein the affinity tag is biotin, c-myc-tag, HA-tag, digoxygenin, flag-tag, dinitrophenol, His tag, strep-tag, glutathione, nickel-nitrilotriacetic acid (NTA), or maltose.
